(19) United States
(12) Patent Application Publication
(10) Pub. No.: US 2022/0008113 A1
(43) Pub. Date: Jan. 13, 2022

(54) SYSTEMS AND METHODS FOR TEMPERATURE CONTROL IN RF ABLATION SYSTEMS

(71) Applicant: Advanced Neuromodulation Systems, Inc., Plano, TX (US)

(72) Inventors: Seil Oh, Dallas, TX (US); Binesh Balasingh, Prosper, TX (US); Christopher Ayers, Dallas, TX (US)

(21) Appl. No.: 16/924,291

(22) Filed: Jul. 9, 2020

(51) Int. Cl. A61B 18/12; A61B 18/14; G16H 40/63; G16H 40/40; G05B 19/4155; G05D 23/19

(52) U.S. Cl. CPC A61B 18/1206; A61B 18/1477; G16H 40/63; A61B 2018/00714; G05B 19/4155; G05D 23/1917; G16H 40/40

(57) ABSTRACT

The present disclosure provides systems and methods for controlling temperature in a radiofrequency (RF) ablation system. A temperature control system includes a subtractor circuit configured to calculate a temperature error as a difference between a target temperature and a measured temperature at a tip of a cannula, and a proportional-integral-derivative (PID) controller coupled to the subtractor circuit and configured to apply an RF voltage to the tip of the cannula, the PID controller configured to determine the RF voltage based on the temperature error and a proportional coefficient, an integral coefficient, and a derivative coefficient of the PID controller. The temperature control system further includes a PID coefficient controller coupled to the PID controller, the PID coefficient controller configured to dynamically adjust the proportional, integral, and derivative coefficients of the PID controller during operation of the RF ablation system.

### A. FIELD OF THE DISCLOSURE

The present disclosure relates generally to radiofrequency (RF) ablation systems, and more particularly to controlling temperatures in RF ablation systems.

### B. BACKGROUND ART

RF nerve ablation may be used, for example, to treat osteoarthritic pain of the spine. Specifically, RF ablation therapy reduces pain through the destruction of nerves using RF energy. The RF energy may be tuned based on cannula size, target temperatures, and dwell time. For example, certain anatomical targets generally require a relatively large cannula to create a relatively large lesion, while other anatomical targets may require a relatively small cannula to limit collateral damage.

At least some known RF ablation systems use a closed-loop control scheme to control RF power to achieve a target temperature without requiring human input. Closed-loop control allows therapy to be relatively straightforward (e.g., as simple as performing an injection).

In an RF ablation system, an RF ablation generator controls operation of a cannula. Different types of RF ablation generators vary in their ability to heat cannulas with different tip sizes. However, cannulas are generally disposable components, and the size of a connected cannula tip is typically not provided to the RF ablation generator via user input or otherwise. As a result, at least some known RF ablation generators include a temperature control system that employs a “one-size fits all” approach. Such systems may perform well for mid-sized cannulas, but may underperform for relatively large and relatively small cannulas. For example, such systems may waste power and fail to heat relatively large cannulas, or may heat relatively small cannulas so fast that suboptimal lesions are generated.

Accordingly, it would be desirable to provide an RF ablation system that includes a temperature control system that automatically compensates for the size of the attached cannula.

### BRIEF SUMMARY OF THE DISCLOSURE

In one embodiment, the present disclosure is directed to a temperature control system for use in a radiofrequency (RF) ablation system including a cannula. The temperature control system includes a subtractor circuit configured to calculate a temperature error as a difference between a target temperature and a measured temperature at a tip of a cannula, and a proportional-integral-derivative (PID) controller coupled to the subtractor circuit and configured to apply an RF voltage to the tip of the cannula, the PID controller configured to determine the RF voltage based on the temperature error and a proportional coefficient, an integral coefficient, and a derivative coefficient of the PID controller. The temperature control system further includes a PID coefficient controller coupled to the PID controller, the PID coefficient controller configured to dynamically adjust the proportional, integral, and derivative coefficients of the PID controller during operation of the RF ablation system.

In another embodiment, the present disclosure is directed to a radiofrequency (RF) ablation system. The RF ablation system includes a cannula including a tip, and an RF ablation generator coupled to the cannula, the RF ablation generator including a temperature control system. The temperature control system includes a subtractor circuit configured to calculate a temperature error as a difference between a target temperature and a measured temperature at a tip of a cannula, and a proportional-integral-derivative (PID) controller coupled to the subtractor circuit and configured to apply an RF voltage to the tip of the cannula, the PID controller configured to determine the RF voltage based on the temperature error and a proportional coefficient, an integral coefficient, and a derivative coefficient of the PID controller. The temperature control system further includes a PID coefficient controller coupled to the PID controller, the PID coefficient controller configured to dynamically adjust the proportional, integral, and derivative coefficients of the PID controller during operation of the RF ablation system.

In another embodiment, the present disclosure is directed to a method of controlling an RF ablation system including a cannula. The method includes calculating a temperature error as a difference between a target temperature and a measured temperature at a tip of the cannula, determining, using a proportional-integral-derivative (PID) controller, an RF voltage based on the temperature error and a proportional coefficient, an integral coefficient, and a derivative coefficient of a PID controller, applying the RF voltage to the tip of the cannula using the PID controller, and dynamically adjusting the proportional, integral, and derivative coefficients of the PID controller during operation of the RF ablation system.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic view of one embodiment of a radiofrequency (RF) ablation system.

FIG. 2 is a diagram of a known temperature control system.

FIGS. 3A and 3B are graphs and that show a simulation of operation of the temperature control system shown in FIG. 2.

FIG. 4 is a diagram of one embodiment of a temperature control system that may be used with the RF ablation system shown in FIG. 1.

FIG. 5 is a flow diagram of one embodiment of a method that may be implemented using the temperature control system shown in FIG. 4.

FIGS. 6A and 6B are graphs comparing operation of the known temperature control system shown in FIG. 2 with the temperature control system shown in FIG. 4.

Corresponding reference characters indicate corresponding parts throughout the several views of the drawings.

### DETAILED DESCRIPTION OF THE DISCLOSURE

The present disclosure provides systems and methods for controlling temperature in a radiofrequency (RF) ablation system. A temperature control system includes a subtractor circuit configured to calculate a temperature error as a difference between a target temperature and a measured temperature at a tip of a cannula. The temperature control system further incudes a proportional-integral-derivative (PID) controller coupled to the subtractor circuit and configured to apply an RF voltage to the tip of the cannula, the PID controller configured to determine the RF voltage based on the temperature error and a proportional coefficient, an integral coefficient, and a derivative coefficient of the PID controller. The temperature control system further includes a PID coefficient controller coupled to the PID controller, the PID coefficient controller configured to dynamically adjust the proportional, integral, and derivative coefficients of the PID controller during operation of the RF ablation system.

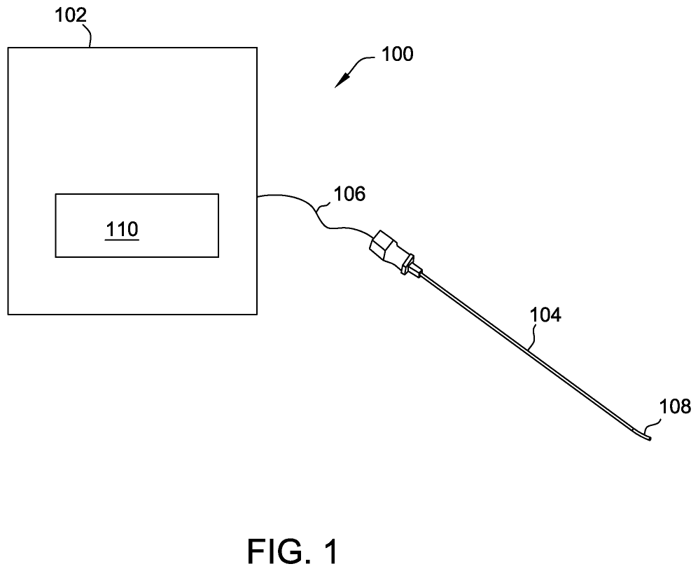

Referring now to the drawings, and in particular to FIG. 1, an RF ablation system is indicated generally at 100. RF ablation system 100 includes an RF ablation generator 102 coupled to a cannula 104 via a cable 106. During operation, RF ablation generator 102 controls energy delivered to a patient through a tip 108 of cannula 104. Further, RF ablation generator 102 includes a temperature control system 110 to facilitate controlling a temperature at tip 108. Specifically, temperature control system 100 attempts to maintain tip 108 at a target temperature.

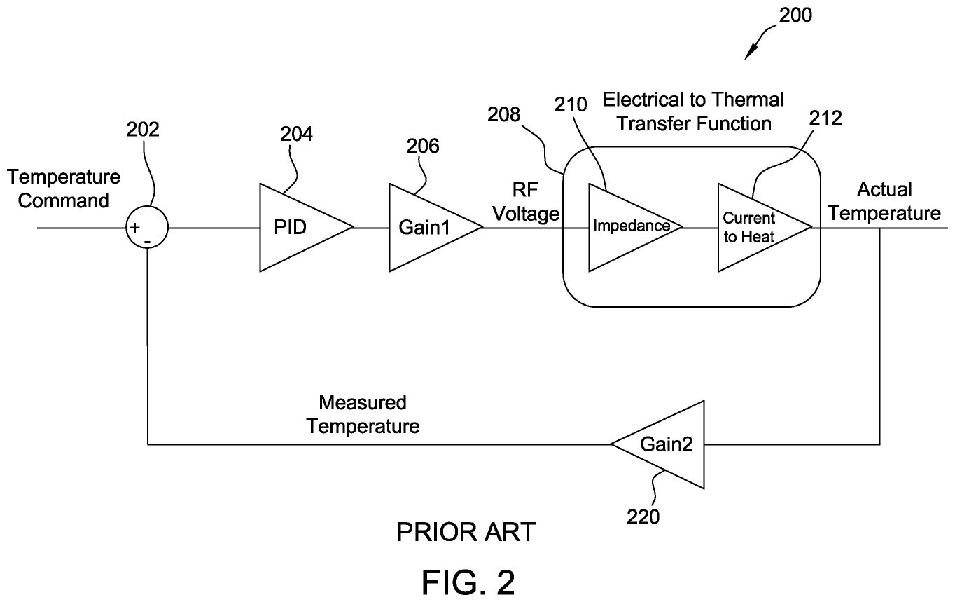

FIG. 2 is a diagram of a known temperature control system 200. Temperature control system 200 may be used with RF ablation system 100. Temperature control system 200 attempts to ramp up to and then maintain a target temperature at tip 108 for a predetermined period of time. The target temperature and predetermined period of time may be set, for example, based on user input received at RF ablation generator 102 (e.g., a target temperature and predetermined period of time input by a clinician).

In the embodiment shown in FIG. 2, temperature control system 200 includes a subtractor circuit 202 that calculates a difference between a temperature command (i.e., the target temperature) and a measured temperature. The calculated difference (also referred to as the temperature error) is supplied to a proportional-integral-derivative (PID) controller 204. As will be appreciated by those of skill in the art, operation of PID controller 204 is controlled by a proportional coefficient (Kp), an integral coefficient (Ki), and a derivative coefficient (Kd). In temperature control system 200, the proportional, integral, and derivative coefficients of PID controller 204 are fixed values.

Based on the calculated difference, PID controller 204 determines an RF voltage (using the proportional, integral, and derivative coefficients), and applies the determined RF voltage to cannula 104 using a first gain circuit 206. More specifically, PID controller 204 attempts to minimize the difference between the temperature command and the measured temperature. First gain circuit 206 may be implemented, for example, using a digital to analog converter (DAC) and RF hardware. Alternatively, first gain circuit 206 may be implemented using any suitable devices.

The applied RF voltage correlates to an actual temperature experienced at tip 108. Specifically, a transfer function 208 dictates the actual temperature that results from a given RF voltage (e.g., based at least in part on an impedance 210 and an electrical current to heat conversion rate 212 of cannula 104).

Every therapy using RF ablation system 100 will have a different transfer function 208 between electrical and thermal energy that PID controller 204 must contend with to attempt to ensure that cannula 104 is properly heated. Specifically, a number of different parameters determined the particular transfer function 208. These parameters include the anatomical target of the therapy (e.g., different therapies require cannula placements in different tissues), the RF accessory used for the therapy (e.g., different therapies require different size cannulas), and the particular patient (e.g., different patients have different electrical and thermal properties).

The actual temperature at tip 108 is measured using a second gain circuit 220 to generate a measured temperature. Second gain circuit 220 may be implemented, for example, using a thermocouple, signal conditioning circuitry, and an analog to digital converter (ADC). Alternatively, second gain circuit 220 may be implemented using any suitable devices. The measured temperature is then provided to subtractor circuit 202, to effect further adjustments by PID controller 204 based on an updated temperature error. Accordingly, temperature control system 200 is a closed-loop control system.

As noted above, temperature control system 200 may perform well for some cannulas, but may underperform for other cannulas (due to variations in the transfer functions).

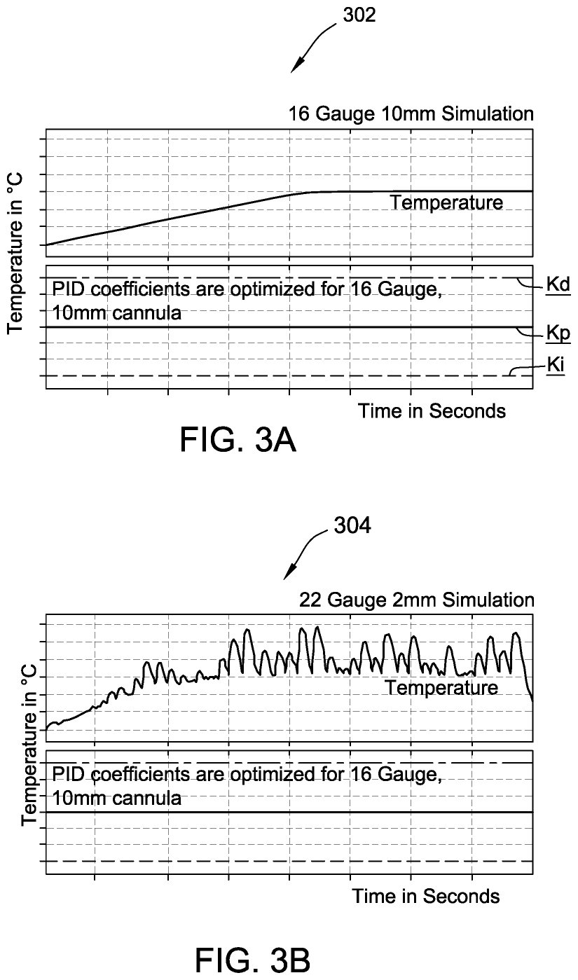

For example, FIGS. 3A and 3B are graphs 302 and 304 that show a simulation of operation of temperature control system 200 when used with a first cannula and a second cannula, respectively. In this example, the first cannula is a 16 gauge cannula with a 10 millimeter (mm) tip, and the second cannula is a 22 gauge cannula with a 2 mm tip. Further, in this simulation, for both cannulas, the goal was to increase the initial temperature to a target temperature at a rate of 3° C./s, and then to maintain the target temperature for proper lesion creation. In addition, in this simulation, the proportional, integral, and derivative coefficients of PID controller 204 were fixed at values optimized for the first cannula.

As shown by graph 302, for the first cannula, the temperature was increased smoothly, and the target temperature was maintained. This is not surprising, given that the proportional, integral, and derivative coefficients of PID controller 204 were fixed at values optimized for the first cannula. However, as shown by graph 304, for the second cannula, there were large oscillations in the temperature both during the increase to the target temperature and while attempting to maintain the target temperature. Accordingly, graphs 302 and 304 demonstrate that PID controller 204 (with fixed coefficients) may perform well for some cannulas, but not for all cannulas.

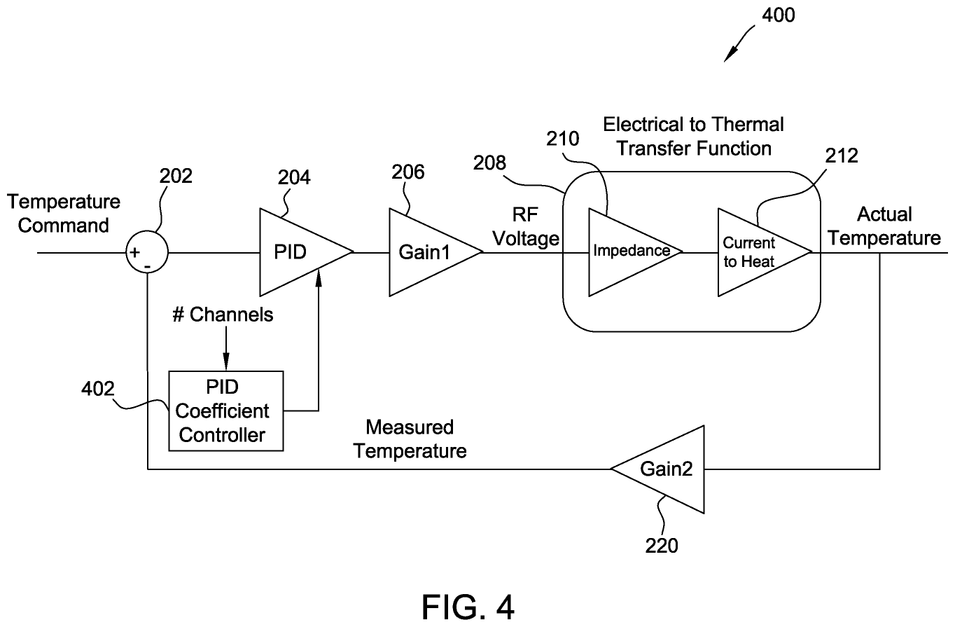

FIG. 4 is a diagram of a temperature control system 400 in accordance with the present disclosure. Temperature control system 400 may be used with RF ablation system 100. Unless otherwise indicated, temperature control system 400 functions similar to temperature control system 200 (shown in FIG. 2), and like elements are labeled with the same reference numeral.

In contrast to temperature control system 200, temperature control system 400 dynamically adjusts the proportional, integral, and derivative coefficients of PID controller 204 during operation to ensure stable performance across a wide range of cannulas. Specifically, as shown in FIG. 4, temperature control system 400 includes a PID coefficient controller 402 that modifies the proportional, integral, and derivative coefficients, as described herein.

By automatically adjusting the control loop during operation, temperature control system 400 enables proper operation of RF ablation system 100 for a plurality of different cannulas. Specifically, PID coefficient controller 402 automatically and dynamically adjusts the proportional, integral, and derivative coefficients to match whatever type of cannula 104 is currently attached to RF ablation generator 102. As compared to known temperature control systems, the temperature control systems described herein facilitate rapidly and reliably heating both large and small cannulas, resulting in a higher rate of therapy success.

In one embodiment, PID coefficient controller 402 initializes the proportional, integral, and derivative coefficients with values for the largest possible cannula size. Alternatively, PID coefficient controller 402 may initialize the coefficients to any suitable values. In example, the coefficients may be initialized as Kp=37.5, Ki=0.75, and Kd=56.25. Alternatively, those of skill in the art will appreciate that the coefficients may be initialized to any suitable value.

The initialized coefficients are then scaled by the number of active channels in RF ablation system 100. The number of active channels corresponds to the number of active electrodes. For example, systems with multiple channels may include multiple active electrodes on a single cannula and/or multiple active cannulas. The number of channels may be determined, for example, based on a user input received at RF ablation generator 102. Alternatively, the number of channels may be automatically determined by RF ablation generator 102 in some embodiments.

In one embodiment, the initialized coefficients are scaled according to the following Table 1:

TABLE 1

| Number of | PID Coefficient |
|---|---|
| Active Channels | Scalars |
| 1 | 1.00 |
| 2 | 1.41 |
| 3 | 1.73 |
| 4 | 2.00 |

Scaling the initialized coefficients ensures that the same amount of power is delivered across multiple channels, due to time-multiplexing schemes implemented by RF ablation generator 102. For example, when four channels are active, the same temperature error will result in doubled output voltages as compared to when only two channels are active.

During operation, PID coefficient controller 402 monitors the calculated temperature error (i.e., the difference between the temperature command and the measured temperature) at a predetermined sampling frequency (e.g., 8 Hz). The temperature error is compared to one or more threshold values, and the coefficients are adjusted accordingly, as described herein in detail.

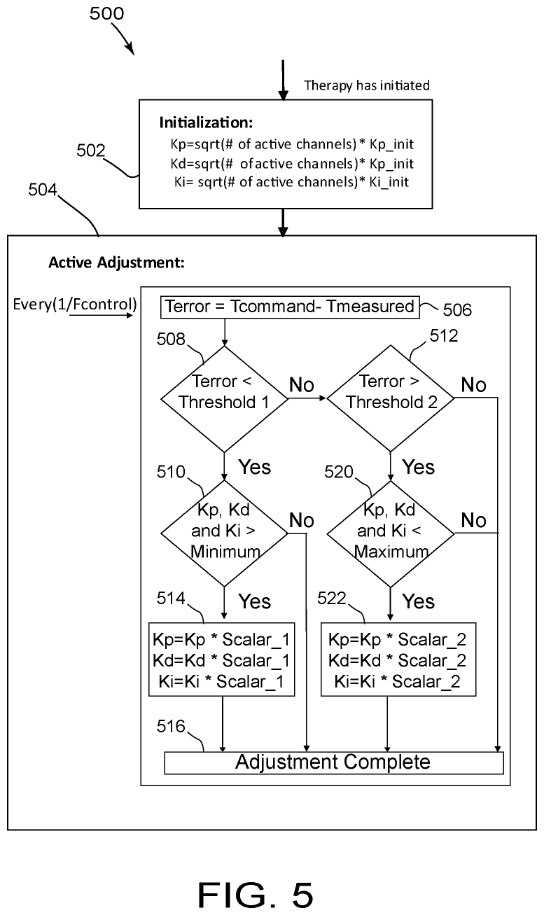

FIG. 5 is a flow diagram illustrating one embodiment of a method 500 for dynamically adjusting the proportional, integral, and derivative coefficients that may be used with temperature control system 400. After a therapy session is initiated, at block 502, the proportional, integral, and derivative coefficients (Kp, Ki, and Kd, respectively) are initialized as discussed above.

As shown in FIG. 5, at block 504, adjustment of the coefficients occurs periodically at a predetermined frequency (i.e., 1/F_{CONTROL}). To adjust the coefficients, the temperature error is calculated at block 506 (i.e., T_{ERROR}=T_{COMMAND}−T_{MEASURED}).

In this embodiment, the temperature error is compared to upper and lower thresholds that define a temperature range. If the temperature error falls outside the temperature range, the coefficients are adjusted accordingly, as described herein.

Specifically, at block 508, the temperature error is compared to the lower threshold. The lower threshold may be, for example, 1° C. If the temperature error is less than the lower threshold, flow proceeds to block 510. If the temperature error is not less than the lower threshold, flow proceeds to block 512.

At block 510, the coefficients are compared to a minimum value (also referred to as a floor value for coefficients). The floor value may be the same for each coefficient, or different coefficients may have different floor values. The floor value may be, for example, 25% of the initial value. If the coefficients are each greater than the floor value, flow proceeds to block 514. If the coefficients are not greater than the floor value, flow proceeds to block 516, and the current adjustment cycle ends (no adjustment is made in this scenario).

At block 514, the coefficients are each reduced by a predetermined amount. Specifically, the coefficients are updated by multiplying each coefficient by a first scalar (Scalar_1). The first scalar may be the same for each coefficient, or different coefficients may be multiplied by different first scalars. The first scalar may be, for example, 0.9 (i.e., reducing the coefficients by 10%). Alternatively, the first scalar may be any suitable value (e.g., 0.75, 0.5, or 0.25). Flow then proceeds to block 516, and the current adjustment cycle ends.

At block 512, the temperature error is compared to the upper threshold. The upper threshold may be, for example, 2° C. If the temperature error is greater than the upper threshold, flow proceeds to block 520. If the temperature error is not greater than the upper threshold (indicating that the temperature error falls within the temperature range), flow proceeds to block 516, and the current adjustment cycle ends.

At block 520, the coefficients are compared to a maximum value (also referred to as a ceiling value for coefficients). The ceiling value may be the same for each coefficient, or different coefficients may have different ceiling values. The ceiling value may be, for example, 110% of the initial value. If the coefficients are each less than the ceiling value, flow proceeds to block 522. If the coefficients are not less than the ceiling value, flow proceeds to block 516, and the current adjustment cycle ends (no adjustment is made in this scenario).

At block 522, the coefficients are each increased by a predetermined amount. Specifically, the coefficients are updated by multiplying each coefficient by a second scalar (Scalar_2). The second scalar may be the same for each coefficient, or different coefficients may be multiplied by different second scalars. The second scalar may be, for example, 1.1 (i.e., increasing the coefficients by 10%). Alternatively, the first scalar may be any suitable value (e.g., 1.25, 1.5, 1.75, or 2.0). Flow then proceeds to block 516, and the current adjustment cycle ends.

Using method 500, the coefficients of PID controller 204 are rapidly scaled and quickly settle to stable values, resulting in stable operation of temperature control system 400

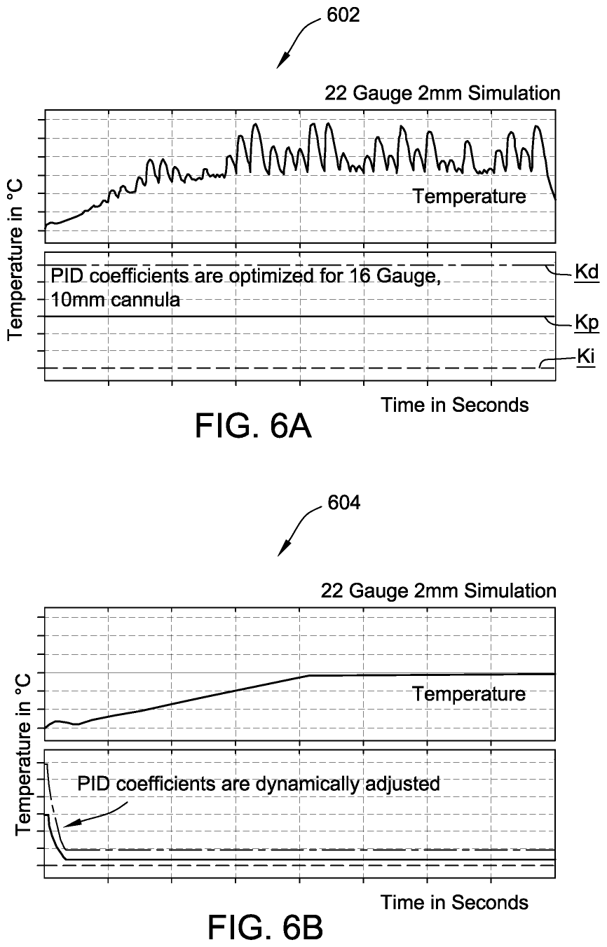

For example, FIGS. 6A and 6B are graphs 602 and 604 comparing operation of temperature control system 200 with temperature control system 400 when used with the second cannula (i.e., a 22 gauge cannula with a 2 mm tip). Again, in these simulations, the goal was to increase the initial temperature to a target temperature at a rate of 3° C./s, and then to maintain the target temperature for proper lesion creation. Graph 602 simulates operation of temperature control system 200 (i.e., with the proportional, integral, and derivative coefficients of PID controller 204 were fixed at values optimized for the first cannula). As shown by graph 602, when using temperature control system 200, there were large oscillations in the temperature both during the increase to the target temperature and while attempting to maintain the target temperature.

In contrast, graph 604 simulates operation of temperature control system 400 using method 500. As shown in FIG. 6B, by dynamically adjusting the coefficients during operation of temperature control system 400, the temperature was increased smoothly, and the target temperature was maintained. Accordingly, graphs 602 and 604 demonstrate that temperature control system 400 provides significant advantages over temperature control system 200.

As compared to at least some known temperature control systems with static PID coefficients (e.g., temperature control system 200), temperature control system 400 dynamically adjusts control loop performance (by adjusting PID coefficients) to improve cannula heating performance. As a result, temperature control system 400 is compatible with a wide range of cannulas without requiring a user input specifying the cannula size. Obviating the need for user input of the cannula size also eliminates the potential for user error.

Thus, the embodiments described herein are able to dynamically adjust PID coefficients as lesioning is performed. In contrast, at least some known systems attempt to characterize the transfer function prior to lesioning, lengthening procedure time. As such, using the systems methods described herein, clinicians (e.g., interventional anesthesiologists) may perform many RF ablations in a relatively short time period, due to the efficient workflow enabled by the temperature control systems described herein.

The embodiments described herein provide systems and methods for controlling temperature in am RF ablation system. A temperature control system includes a subtractor circuit configured to calculate a temperature error as a difference between a target temperature and a measured temperature at a tip of a cannula. The temperature control system further incudes a PID controller coupled to the subtractor circuit and configured to apply an RF voltage to the tip of the cannula, the PID controller configured to determine the RF voltage based on the temperature error and a proportional coefficient, an integral coefficient, and a derivative coefficient of the PID controller. The temperature control system further includes a PID coefficient controller coupled to the PID controller, the PID coefficient controller configured to dynamically adjust the proportional, integral, and derivative coefficients of the PID controller during operation of the RF ablation system.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A temperature control system for use in a radiofrequency (RF) ablation system including a cannula, the temperature control system comprising:
a subtractor circuit configured to calculate a temperature error as a difference between a target temperature and a measured temperature at a tip of the cannula;
a proportional-integral-derivative (PID) controller coupled to the subtractor circuit and configured to apply an RF voltage to the tip of the cannula, the PID controller configured to determine the RF voltage based on the temperature error and a proportional coefficient, an integral coefficient, and a derivative coefficient of the PID controller; and
a PID coefficient controller coupled to the PID controller, the PID coefficient controller configured to dynamically adjust the proportional, integral, and derivative coefficients of the PID controller during operation of the RF ablation system.

2. The temperature control system of claim 1, wherein to dynamically adjust the proportional, integral, and derivative coefficients, the PID coefficient controller is configured to dynamically adjust the proportional, integral, and derivative coefficients based on the temperature error.

3. The temperature control system of claim 2, wherein to dynamically adjust the proportional, integral, and derivative coefficients, the PID coefficient controller is configured to decrease the proportional, integral, and derivative coefficients when the measured temperature is below a lower threshold temperature.

4. The temperature control system of claim 2, wherein to dynamically adjust the proportional, integral, and derivative coefficients, the PID coefficient controller is configured to increase the proportional, integral, and derivative coefficients when the measured temperature is above an upper threshold temperature.

5. The temperature control system of claim 2, wherein to dynamically adjust the proportional, integral, and derivative coefficients, the PID coefficient controller is configured to maintain the proportional, integral, and derivative coefficients when the measured temperature is within a temperature range defined by a lower threshold temperature and an upper threshold temperature.

6. The temperature control system of claim 2, wherein to dynamically adjust the proportional, integral, and derivative coefficients, the PID coefficient controller is configured to decrease the proportional, integral, and derivative coefficients when i) the measured temperature is below a lower threshold temperature and ii) the proportional, integral, and derivative coefficients are above a floor value.

7. The temperature control system of claim 2, wherein to dynamically adjust the proportional, integral, and derivative coefficients, the PID coefficient controller is configured to increase the proportional, integral, and derivative coefficients when i) the measured temperature is above an upper threshold temperature and ii) the proportional, integral, and derivative coefficients are below a ceiling value.

8. A radiofrequency (RF) ablation system comprising:
a cannula comprising a tip; and
an RF ablation generator coupled to the cannula, the RF ablation generator comprising a temperature control system that comprises:
a subtractor circuit configured to calculate a temperature error as a difference between a target temperature and a measured temperature at a tip of the cannula;
a proportional-integral-derivative (PID) controller coupled to the subtractor circuit and configured to apply an RF voltage to the tip of the cannula, the PID controller configured to determine the RF voltage based on the temperature error and a proportional coefficient, an integral coefficient, and a derivative coefficient of the PID controller; and
a PID coefficient controller coupled to the PID controller, the PID coefficient controller configured to dynamically adjust the proportional, integral, and derivative coefficients of the PID controller during operation of the RF ablation system.

9. The RF ablation system of claim 8, wherein to dynamically adjust the proportional, integral, and derivative coefficients, the PID coefficient controller is configured to dynamically adjust the proportional, integral, and derivative coefficients based on the temperature error.

10. The RF ablation system of claim 9, to dynamically adjust the proportional, integral, and derivative coefficients, the PID coefficient controller is configured to decrease the proportional, integral, and derivative coefficients when the measured temperature is below a lower threshold temperature.

11. The RF ablation system of claim 9, wherein to dynamically adjust the proportional, integral, and derivative coefficients, the PID coefficient controller is configured to increase the proportional, integral, and derivative coefficients when the measured temperature is above an upper threshold temperature.

12. The RF ablation system of claim 9, wherein to dynamically adjust the proportional, integral, and derivative coefficients, the PID coefficient controller is configured to maintain the proportional, integral, and derivative coefficients when the measured temperature is within a temperature range defined by a lower threshold temperature and an upper threshold temperature.

13. The RF ablation system of claim 9, wherein to dynamically adjust the proportional, integral, and derivative coefficients, the PID coefficient controller is configured to decrease the proportional, integral, and derivative coefficients when i) the measured temperature is below a lower threshold temperature and ii) the proportional, integral, and derivative coefficients are above a floor value.

14. The RF ablation system of claim 9, wherein to dynamically adjust the proportional, integral, and derivative coefficients, the PID coefficient controller is configured to increase the proportional, integral, and derivative coefficients when i) the measured temperature is above an upper threshold temperature and ii) the proportional, integral, and derivative coefficients are below a ceiling value.

15. A method of controlling an RF ablation system including a cannula, the method comprising:
calculating a temperature error as a difference between a target temperature and a measured temperature at a tip of the cannula;
determining, using a proportional-integral-derivative (PID) controller, an RF voltage based on the temperature error and a proportional coefficient, an integral coefficient, and a derivative coefficient of a PID controller;
applying the RF voltage to the tip of the cannula using the PID controller; and
dynamically adjusting the proportional, integral, and derivative coefficients of the PID controller during operation of the RF ablation system.

16. The method of claim 15, wherein dynamically adjusting the proportional, integral, and derivative coefficients comprises dynamically adjusting the proportional, integral, and derivative coefficients based on the temperature error.

17. The method of claim 16, wherein dynamically adjusting the proportional, integral, and derivative coefficients comprises decreasing the proportional, integral, and derivative coefficients when the measured temperature is below a lower threshold temperature.

18. The method of claim 16, wherein dynamically adjusting the proportional, integral, and derivative coefficients comprises increasing the proportional, integral, and derivative coefficients when the measured temperature is above an upper threshold temperature.

19. The method of claim 16, wherein dynamically adjusting the proportional, integral, and derivative coefficients comprises maintaining the proportional, integral, and derivative coefficients when the measured temperature is within a temperature range defined by a lower threshold temperature and an upper threshold temperature.

20. The method of claim 16, wherein dynamically adjusting the proportional, integral, and derivative coefficients comprises decreasing the proportional, integral, and derivative coefficients when i) the measured temperature is below a lower threshold temperature and ii) the proportional, integral, and derivative coefficients are above a floor value.
